(19) United States
(12) Patent Application Publication
(10) Pub. No.: US 2011/0196407 A1
(43) Pub. Date: Aug. 11, 2011

(54) MULTIPLE FUNCTION SURGICAL INSTRUMENT

(76) Inventor: Moutaa BenMaamer, Ft. Myers, FL (US)

(21) Appl. No.: 13/020,239

(22) Filed: Feb. 3, 2011

Related U.S. Application Data
(60) Provisional application No. 61/301,823, filed on Feb. 5, 2010.

(51) Int. Cl. A61B 17/32; A61B 17/34

(52) U.S. Cl. 606/174; 606/185

(57) ABSTRACT

An instrument used in laparoscopic surgery is disclosed, the instrument having a trocar having a hollow interior and a sharpened end, and at least two implements having working distal ends and associated shafts extending from the distal ends and terminating in respective handles at proximal ends thereof disposed within the hollow interior of the trocar, wherein the implements may be selectively advanced and retracted by an appropriate force applied to the respective handles of the implements.

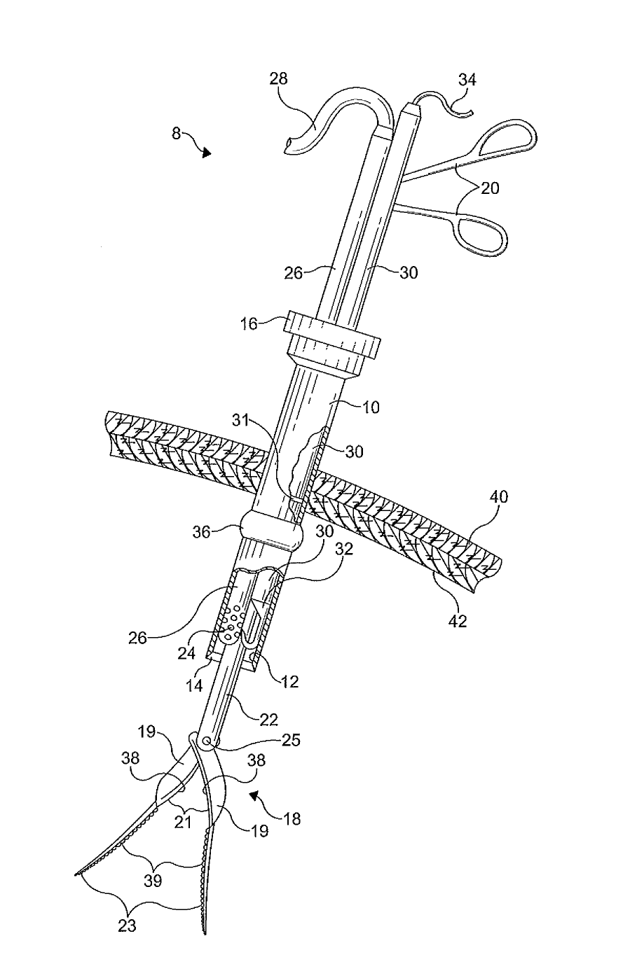

### CROSS-REFERENCE TO RELATED APPLICATION

This application claims the benefit of priority of U.S. Provisional Patent Application Ser. No. 61/301,823 filed on Feb. 5, 2010 hereby incorporated herein by reference in its entirety.

### FIELD OF THE INVENTION

The present invention relates to an implement for performing minimally invasive surgery and, more particularly, to a multiple function implement for performing a laparoscopic surgery.

### BACKGROUND OF THE INVENTION

Laparoscopic surgery, or laparoscopy, is a minimally invasive surgery inside the abdominal cavity, chest cavity, and/or the joints of a patient, for example. During a typical laparoscopy, the desired cavity of the patient is insufflated, if necessary, with gas, and cannula sleeves, or trocars, are passed through small incisions (approximately one-half inch) to provide entry ports for receiving laparoscopic surgical instruments.

The laparoscopic instruments include a laparoscope (for viewing the surgical field) and other separate instruments for performing a number of different tasks. These instruments are similar to those used in a conventional surgery, except the working end, or effector, of each instrument is spaced from its respective handle by an extension tube. The effector of each instrument may include clamps, graspers or dissectors, scissors, suction or irrigation tubes, and needle holders, for example. The surgeon guides a desired instrument, such as one of the above instruments, through a cannula sleeve to an internal surgical site. The desired instrument is manipulated from an outside of the abdominal cavity. The surgeon observes the procedure by means of a monitor that displays an image of the surgical site taken from the laparoscope.

Minimally invasive surgical systems are being developed to overcome some of the disadvantages of open surgeries. Minimally invasive surgical instruments deny the surgeon the flexibility of implement placement found in open surgery. Most current laparoscopic implements have rigid shafts. The rigid shafts may create difficulty in approaching the planned surgical site through a small incision. The length of many instruments reduces the ability of a surgeon to feel forces exerted by tissues and organs on the end effector of the implement that is in use. The lack of dexterity and sensitivity, coupled with the necessity to lose some concentration during the change from one instrument to another. Also, every instrument has only one function, and this is a major impediment to the expansion of minimally invasive surgery.

It would be desirable to produce an instrument for use during minimally invasive surgery that will minimize or eliminate the time required between the change of surgical functions of the instrument.

### SUMMARY OF THE INVENTION

Concordant and congruous with the present invention, an instrument for use during minimally invasive surgery that will minimize the time required between the change of surgical functions of the instrument has surprisingly been discovered.

In an embodiment of the invention, An instrument used in laparoscopic surgery comprises a trocar having a hollow interior and a sharpened end; and at least two implements having working distal ends and associated shafts extending from the distal ends and terminating in respective handles at proximal ends thereof disposed within the hollow interior of the trocar, wherein the implements may be selectively advanced and retracted by an appropriate force applied to the respective handles of the implements.

In another embodiment of the invention, An instrument used in laparoscopic surgery comprises a trocar having a hollow interior and a sharpened end; and at least three implements having working distal ends and associated shafts extending from the distal ends and terminating in respective handles at proximal ends thereof, wherein the implements are frictionally engaged within the hollow interior of the trocar and may be selectively advanced and retracted by an appropriate force applied to the respective handles of the implements.

### BRIEF DESCRIPTION OF THE DRAWINGS

The above, as well as other objects and advantages of the invention, will become readily manifest to those skilled in the art from reading the following detailed description when considered in light of the attached drawing in which the FIGURE is an elevational view, partly in section, of a multifunctional instrument for use in minimally invasive surgery embodying the features of the present invention.

### DETAILED DESCRIPTION OF THE PREFERRED EMBODIMENT OF THE INVENTION

The following detailed description and appended drawings describe and illustrate various exemplary embodiments of the invention. The description and drawings serve to enable one skilled in the art to make and use the invention, and are not intended to limit the scope of the invention in any manner. In respect of the methods disclosed, the steps presented are exemplary in nature, and thus, the order of the steps is not necessary or critical.

Referring to the drawing, there is shown a multiple-function surgical instrument 8 including a trocar 10 having a hollow interior 12 and a sharpened distal end edge 14. The proximal end 16 of the trocar 10 is designed to typically receive a number of individual implements for use in surgical procedures. The proximal end 16 includes a gasket or a seal to provide a seal between the trocar 10 and the individual implements. In the FIGURE, the implements include a dissector/scissors implement 18 equipped with an operating looped handle 20 at the proximal end of a shaft 22. The dissector/scissors implement 18 is disposed at the distal or working end of the shaft 22.

The dissector/scissors implement 18 includes a pair of opposing pivotally-connected members 19. Each of the pair of members 19 includes a first portion 21 for cutting and a second portion 23 for grasping. The first portions 21 of the members 19 are disposed closest to a pivotal point 25 of the dissector/scissors implement 18. Each of the first portions 21 includes blades 38. Each of the second portions 23 includes ridges or serrations 39.

Another implement within the trocar 10 is a perforated irrigator/suction implement 24 at the distal end of a hollow shaft 26. The proximal end of the shaft 26 is coupled to a source of irrigation or suction (not shown) as determined by the surgeon, typically through a tube 28.

A third implement is included in the illustrated embodiment that consists of a shaft 30 having a hook implement 32 at a distal end thereof and an electrical lead 34 at a proximal end thereof. It will be understood that the hook implement 32 is electrically coupled to the electrical lead 34, which, in turn, is connected to a source of electrical energy (not shown).

To militate against undesired contact between the implements 18, 24, 32, each implement 18, 24, 32, and each associated shaft 22, 26, 30, respectively, may be housed in a retractable sheath (not shown). The sheaths of each implement 18, 24, 32 may be retracted or advanced by manipulation of each sheath at a proximal end of the multiple-function surgical instrument 8.

It will be understood that in use, the implements 18, 24, 32 are disposed within the trocar 10 in a retracted position, as the irrigator/suction implement 24 and the hook implement 32 are illustrated in the FIGURE. The sharpened end edge 14 of the trocar 10 may then be inserted into the abdominal cavity of a patient, typically through a small incision in the abdominal cavity, causing the end edge 14 of the trocar 10 to enter the abdominal cavity of the patient through the abdominal wall, comprised of skin 40 and the adjacent muscle 42.

The implements 18, 24, 32 are frictionally engaged within the hollow interior of the trocar 10. The implements 18, 24, 32 may be selectively advanced and retracted by an appropriate force applied by the surgeon to the proximal end of the desired implement 18, 24, 32. In the event it is deemed necessary or desirable to vary the frictional engagement of the implements 18, 24, 32 within the trocar 10, a flexible member 31 may be inserted to extend around the shafts of the implements 18, 24, 32. The flexible member 31 may be gasket or ring disposed around or formed on the shafts of the implements 18, 24, 32, or the flexible member 31 may be disposed around or formed on each shaft individually. It is understood that the flexible member 31 may be disposed around any combination of the implements 18, 24, 32, as desired. The flexible member 31 may be any member adapted to elastically deform when disposed on the trocar 10, such as an annular spring, for example.

As shown in the FIGURE, a portion of an exterior of the trocar 10 includes a selectively expandable member 36 disposed thereon adjacent the distal end thereof. The expandable member 36 is formed from an elastic material adapted to be inflated or expanded. Once the expandable member 36 of the trocar 10 is inserted into the abdominal cavity of the patient, the expandable member 36 may be elastically expanded to militate against the removal of the trocar 10 from the abdominal cavity during a procedure. A source of fluid (not shown), such as a hand pump, in communication with the expandable member 36 may be used to selectively inflate and deflate the expandable member 36. It is understood that the fluid used to inflate the expandable member 36 may be a gas or a liquid, such as a saline solution, for example, as desired.

It will be appreciated that the afore-described instrument 8 may incorporate a number of different implements readily available for selective use by the surgeon without requiring a request to an accompanying nurse or other medical provider for another implement, thereby allowing the surgeon's concentration to be continuous without a break, resulting in increased efficiency and reduced operating time. Additional implements incorporated into the multiple-function surgical instrument 8 may include clamps, needle holders, a camera, and a light, for example. The consequence results in manifest benefits to the patient.

From the foregoing description, one ordinarily skilled in the art can easily ascertain the essential characteristics of this invention and, without departing from the spirit and scope thereof, can make various changes and modifications to the invention to adapt it to various usages and conditions.

What is claimed is:

1. An instrument used in laparoscopic surgery comprising:
a trocar having a hollow interior and a sharpened end; and
a pair of implements having working distal ends and associated shafts extending between distal ends and proximal ends thereof disposed within the hollow interior of the trocar, wherein the implements are selectively advanced and retracted by an appropriate force applied to the respective handles of the implements.

2. The instrument of claim 1, wherein one of the implements is a dissector/scissors implement having a pair of pivotally connected blades.

3. The instrument of claim 2, wherein the dissector/scissors implement also have a pair of grasping portions formed adjacent the pivotally connected blades.

4. The instrument of claim 1, wherein an other of the implements is one of a hook implement and an irrigator/suction implement.

5. The instrument of claim 4, wherein the other of the implements is a hook implement disposed on the distal end of the associated shaft, the shaft having an electrical lead in electrical communication with a source of electricity disposed on the proximal end thereof.

6. The instrument of claim 1, further comprising a third implement having a working distal end and a shaft extending from the distal end and terminating in a handle at a proximal end thereof, wherein the third implement may be selectively advanced and retracted by an appropriate force applied to the handle thereof.

7. The instrument of claim 1, wherein the implements are frictionally engaged within the hollow interior of the trocar.

8. The instrument of claim 1, further comprising a flexible member extending around the shaft of at least one of the implements to increase a frictional engagement of the implements within the hollow interior of the trocar.

9. The instrument of claim 1, further comprising a selectively expandable member disposed on a distal end of the trocar.

10. The instrument of claim 1, wherein a proximal end of the trocar includes a seal for providing a seal between the trocar and the implements.

11. An instrument used in laparoscopic surgery comprising:
a trocar having a hollow interior and a sharpened end; and
at least three implements having working distal ends and associated shafts extending between distal ends and proximal ends thereof, wherein the implements are frictionally engaged within the hollow interior of the trocar and may be selectively advanced and retracted by an appropriate force applied to the respective handles of the implements.

12. The instrument of claim 11, wherein one of the implements is a dissector/scissors implement having a pair of pivotally connected blades.

13. The instrument of claim 12, wherein the dissector/scissors implement also have a pair of grasping portions formed adjacent the pivotally connected blades.

14. The instrument of claim 11, wherein another of the implements is an irrigator/suction implement.

15. The instrument of claim 11, wherein another of the implements is a hook implement disposed on the distal end of the associated shaft, the shaft having an electrical lead in electrical communication with a source of electricity disposed on the proximal end thereof.

16. The instrument of claim 11, further comprising a flexible member extending around the shaft of at least one of the implements to increase a frictional engagement of the implements within the hollow interior of the trocar.

17. The instrument of claim 11, further comprising a selectively expandable member disposed on a distal end of the trocar.

18. The instrument of claim 11, wherein a proximal end of the trocar includes a seal for providing a seal between the trocar and the implements.
